(19) United States
(12) Patent Application Publication
(10) Pub. No.: US 2016/0228004 A1
(43) Pub. Date: Aug. 11, 2016

(54) Infant body temperature and sleep monitoring system

(71) Applicants: Shu YU, SHENZHEN (CN); Jianhua LIANG, SHENZHEN (CN); Jin HUANG, SHENZHEN (CN)

(72) Inventors: Shu YU, SHENZHEN (CN); Jianhua LIANG, SHENZHEN (CN); Jin HUANG, SHENZHEN (CN)

(21) Appl. No.: 14/698,864

(22) Filed: Apr. 29, 2015

(30) Foreign Application Priority Data
Feb. 11, 2015 (CN) 201520098274.9
Feb. 11, 2015 (CN) 201520103505.0

(51) Int. Cl. A61B 5/00; A61B 5/01

(52) U.S. Cl. CPC A61B 5/0024; A61B 5/01; A61B 5/0008; A61B 5/6824; A61B 5/6831; A61B 5/4806; A61B 2503/04; A61B 2560/045; A61B 2562/0219

(57) ABSTRACT

The present invention relates to an infant body temperature and sleep monitoring system including a Bluetooth body temperature thermometer main body, an arm belt and a Bluetooth receiving member; the Bluetooth body temperature thermometer main body is connected to a first side of the arm belt via a first adhesive strap; a second side of the arm belt is disposed with a fastening ring at a first end and a second adhesive strap at a second end; the second end of the arm belt which is disposed with the second adhesive strap passes through the fastening ring and engages with the second adhesive strap so that the arm belt forms a tightness-adjustable annular belt; the Bluetooth body temperature thermometer main body obtains body temperature data and transmits the body temperature data to the Bluetooth receiving member. The present invention is simple in structure, convenient and practical and easy to wear.

### TITLE OF THE INVENTION

An infant body temperature and sleep monitoring system

### BACKGROUND OF THE INVENTION

The present invention relates to monitoring equipment and more particularly pertains to an infant body temperature and sleep monitoring system.

When using an existing thermometer to measure body temperature of an infant or baby, as the infant or baby could not actively cooperate, it is usually difficult to measure the real body temperature. Infants and babies are incapable of informing adults about variations of their own body temperatures, and adults could not stay with the infants or babies to measure their body temperatures all the time, thus variations in the body temperatures of the infants or babies often could not be detected in time, and effective treatment of diseases of the infants or babies could not be provided in time. The body temperature monitoring products in the marketplace could only monitor body temperature, but could not accurately monitor sleeping positions and are also impossible to disassemble for cleaning.

### BRIEF SUMMARY OF THE INVENTION

In view of the aforesaid disadvantages now present in the prior art, the present invention provides an infant body temperature and sleeping position monitoring system with a disengageable main body.

To attain this, the present invention generally comprises a Bluetooth body temperature thermometer main body, an arm belt and a Bluetooth receiving member; the Bluetooth body temperature thermometer main body is connected to a first side of the arm belt via a first adhesive strap; a second side of the arm belt is disposed with a fastening ring at a first end and a second adhesive strap at a second end; the second end of the arm belt which is disposed with the second adhesive strap passes through the fastening ring and engages with the second adhesive strap so that the arm belt forms a tightness-adjustable annular belt; the Bluetooth body temperature thermometer main body obtains body temperature data and transmits the body temperature data to the Bluetooth receiving member.

Furthermore, the first adhesive strap comprises a first adhesive strap hook side and a first adhesive strap loop side; the first adhesive strap hook side is disposed on a back side of the main body, and the first adhesive strap loop side is disposed on a corresponding position on the arm belt. This facilitates the Bluetooth body temperature thermometer main body to be disengaged from the arm belt for convenient replacement or cleaning of the arm belt.

Furthermore, the second adhesive strap comprises a second adhesive strap hook side and a second adhesive strap loop side; the second adhesive strap hook side is disposed adjacent to the second adhesive strap loop side; the second adhesive strap hook side is disposed at an outer side which facilitates the arm belt to be secured on an arm accurately and for easier tightness adjustment.

Furthermore, the Bluetooth body temperature thermometer main body comprises a Bluetooth module, a temperature sensor, an accelerometer, a Bluetooth antenna and a battery; the temperature sensor, the accelerometer, the Bluetooth antenna and the battery are respectively electrically connected to the Bluetooth module; the temperature sensor and the accelerometer transmit measuring data obtained to the Bluetooth module; the Bluetooth module processes the measuring data obtained and then transmits the processed measuring data to the Bluetooth receiving member via the Bluetooth antenna; the battery supplies power to the Bluetooth module.

Furthermore, the first side of the arm belt is disposed with a pocket for receiving the Bluetooth antenna, so that the Bluetooth antenna is prevented from contacting skin and thus enhance comfort.

Furthermore, the Bluetooth antenna is connected to the Bluetooth module via an RF connector. The RF connector is a mechanical-electrical product mainly for linkage purpose.

Furthermore, the battery is removable battery which is convenient for replacement.

Furthermore, the battery is button battery.

Furthermore, the Bluetooth body temperature thermometer main body further comprises a main body outer casing; the Bluetooth module, the accelerometer and the battery are disposed inside the main body outer casing; the temperature sensor has a probe which is disposed on an outer side of the main body outer casing; the Bluetooth antenna extends from interior of the main body outer casing to exterior of the main body outer casing.

Furthermore, the Bluetooth body temperature thermometer main body has a height of 5-10 mm which is suitable for wear by infants and babies.

Furthermore, the arm belt is made of highly permeable Nylex fabric for medical use, so as to guarantee air permeability and comfort during wear.

The present invention is simple in structure, convenient and practical and easy to wear. The Bluetooth body temperature thermometer main body is disengageable which facilitates cleaning of the arm belt. The Bluetooth body temperature thermometer main body is secured on the arm via the arm belt for sensing body temperature and acceleration data of an infant and transmitting the data to the Bluetooth receiving member, so that guardians may directly perceive the variations in the body temperature and sleeping position of the infant, thus facilitating the guardians to further monitor the sleeping and health status of the infant and to better take care of the infant.

### BRIEF DESCRIPTION OF THE DRAWINGS

FIG. 1 shows the structure of the present invention.

FIG. 2 shows the structures of the Bluetooth body temperature thermometer main body and the arm belt of the present invention.

FIG. 3 shows a rear view of the Bluetooth body temperature thermometer main body of the present invention.

FIG. 4 shows the structure of the second side of the arm belt of the present invention.

FIG. 5 is a disassembling view of the Bluetooth body temperature thermometer main body and the arm belt of the present invention.

FIG. 6 is a circuit block diagram of the present invention.

In the drawings: 1—Bluetooth body temperature thermometer main body; 11—Bluetooth module; 12—Temperature sensor; 13—Accelerometer; 14—Bluetooth antenna; 15—Battery; 16—Main body outer casing; 2—Arm belt; 3—Bluetooth receiving member; 4—First adhesive strap; 41—First adhesive strap hook side; 42—First adhesive strap loop side; 5—Fastening ring; 6—Second adhesive strap; 61—Second adhesive strap hook side; 62—Second adhesive strap loop side; 7—Pocket.

### DETAILED DESCRIPTION OF THE INVENTION

The present invention is further illustrated with the accompanying drawings and an embodiment.

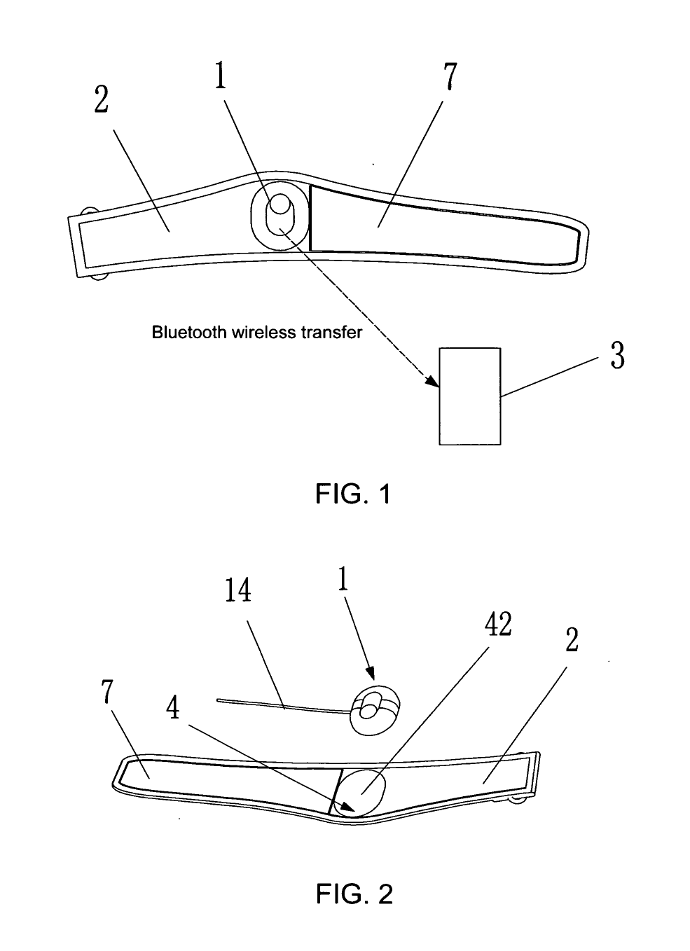

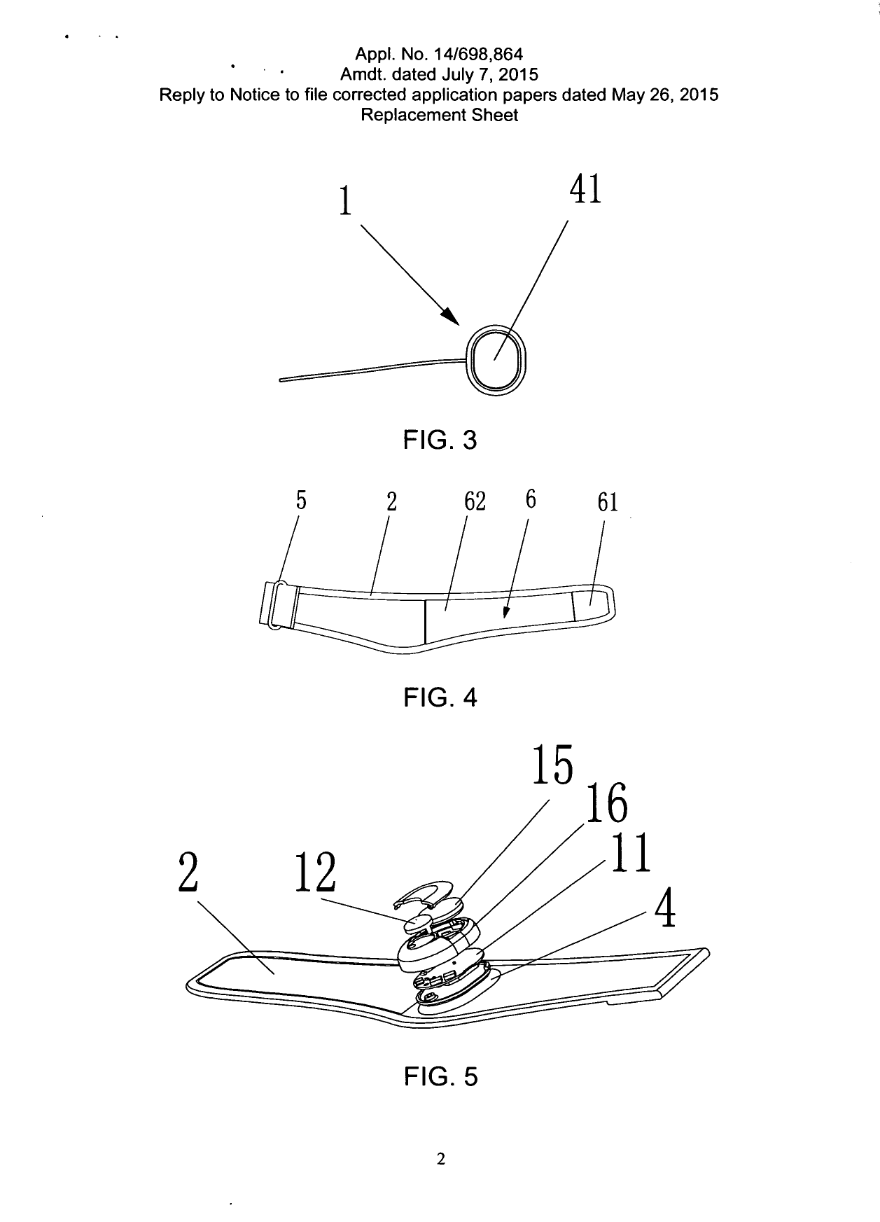

As illustrated in FIGS. 1-5, the infant body temperature and sleep monitoring system of the present invention mainly comprises a Bluetooth body temperature thermometer main body 1, an arm belt 2 and a Bluetooth receiving member 3. The Bluetooth body temperature thermometer main body 1 and the arm belt 2 form a disengageable Bluetooth body temperature thermometer. The Bluetooth body temperature thermometer main body 1 is connected to a first side of the arm belt 2 via a first adhesive strap 4. The first adhesive strap 4 comprises a first adhesive strap hook side 41 and a first adhesive strap loop side 42. The first adhesive strap hook side 41 is disposed on a back side of the main body 1, and the first adhesive strap loop side 42 is disposed on a corresponding position on the arm belt 2 for adhering to the Bluetooth body temperature thermometer main body 1. The Bluetooth body temperature thermometer main body 1 is positioned on the arm belt 2 such that the Bluetooth body temperature thermometer main body 1 is adjacent to armpit of the user after wear. A second side of the arm belt 2 is disposed with a fastening ring 5 at a first end and a second adhesive strap 6 at a second end. The second adhesive strap 6 comprises a second adhesive strap hook side 61 and a second adhesive strap loop side 62. The second adhesive strap hook side 61 is disposed at an outermost side of the second end of the second side of the arm belt 2. The second adhesive strap loop side 62 is disposed adjacent to the second adhesive strap hook side 61 at an inner side. When in use, the end of the arm belt 2 disposed with the second adhesive strap 6 passes through the fastening ring 5, turns around a side of the fastening ring 5, and is then pulled back until a suitable degree of tightness is achieved, and at this time the second adhesive strap hook side 61 is engaged with the second adhesive strap loop side 62, rendering the arm belt 2 to become a rapidly tightness-adjustable annular belt. Preferably, the fastening ring 5 is electrically plated with metals and does not expose to the first side of the arm belt 2 so as to prevent direct contact with skin.

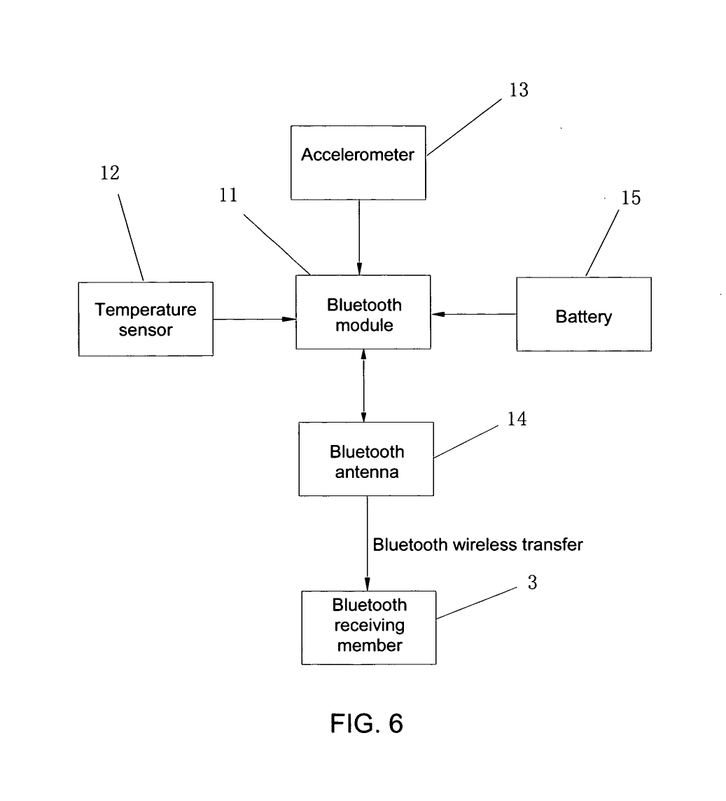

As illustrated in FIG. 6, the Bluetooth body temperature thermometer main body 1 comprises a Bluetooth module 11, a temperature sensor 12, an accelerometer 13, a Bluetooth antenna 14, a battery 15 and a main body outer casing 16. The temperature sensor 12, the accelerometer 13, the Bluetooth antenna 14 and the battery 15 are respectively electrically connected to the Bluetooth module 11. The temperature sensor 12 and the accelerometer 13 transmit measuring data obtained to the Bluetooth module 11. The temperature sensor 12 is used for collecting human body temperature data in real time. The accelerometer 13 is used for detecting human body gravitational acceleration data in real time to determine sleeping position of the infant. The Bluetooth module 11 processes the measuring data obtained and then transmits the processed measuring data to the Bluetooth receiving member 3 via the Bluetooth antenna 14. The battery 15 supplies power to the Bluetooth module 11. Preferably, the battery 15 is removable battery, particularly a button battery. The Bluetooth module 11 and the accelerometer 13 are welded on a PCBA. The temperature sensor 12 is connected to the PCBA via wire. The Bluetooth antenna 14 is connected to the Bluetooth circuit on the PCBA via an RF connector. The Bluetooth module 11, the accelerometer 13 and the battery 15 are disposed inside the main body outer casing 16. The temperature sensor 12 has a probe which is disposed on an outer side of the main body outer casing 16. The Bluetooth antenna 14 extends from interior of the main body outer casing 16 to exterior of the main body outer casing 16.

Preferably, the Bluetooth receiving member 3 is smart terminals such as mobile phones, tablet computers and notebook computers.

Preferably, the first side of the arm belt 2 is disposed with an elongated pocket 3 for receiving the Bluetooth antenna 14 so as to prevent the Bluetooth antenna 14 to injure the skin of the infant or baby.

The armpit of a human is 12-50 mm away from the skeleton. Preferably, the main body 1 has a height of 5-10 mm. In the present embodiment, the height is 8.5 mm so that it could be comfortably worn by babies. The height referred herein is the distance between the highest point and the lowest point of the Bluetooth body temperature thermometer main body 1.

Preferably, the arm belt 2 is made of highly permeable Nylex fabric for medical use and is specially cut so as to guarantee air permeability, comfort and accurate positioning.

When wearing the Bluetooth thermometer, first position the Bluetooth body temperature thermometer main body 1 adjacent to the armpit of the infant, then wrap the arm belt 2 around the arm, and pass the second adhesive strap 6 through the fastening ring 5 to adjust the degree of tightness, and thereafter secure in position by engaging the second adhesive strap hook side 61 with the second adhesive strap loop side 62. The Bluetooth body temperature thermometer main body 1 senses body temperature and sleeping position data via the temperature sensor 12 and the accelerometer 13 and transmits the data to the Bluetooth receiving member 3 via the Bluetooth antenna 14. The Bluetooth receiving member 3 is provided with a client terminal for displaying and managing the infant body temperature and sleeping position data to further monitor the sleeping status of the infant.

A person skilled in the art may make other corresponding modifications and changes based on the foregoing, and such modifications and changes fall within the scope of the claims of the present invention.

What is claimed is:

1. An infant body temperature and sleep monitoring system comprising a Bluetooth body temperature thermometer main body, an arm belt and a Bluetooth receiving member; the Bluetooth body temperature thermometer main body is connected to a first side of the arm belt via a first adhesive strap; a second side of the arm belt is disposed with a fastening ring at a first end and a second adhesive strap at a second end; the second end of the arm belt which is disposed with the second adhesive strap passes through the fastening ring and engages with the second adhesive strap so that the arm belt forms a tightness-adjustable annular belt; the Bluetooth body temperature thermometer main body obtains body temperature data and transmits the body temperature data to the Bluetooth receiving member.

2. The infant body temperature and sleep monitoring system as in claim 1, wherein the first adhesive strap comprises a first adhesive strap hook side and a first adhesive strap loop side; the first adhesive strap hook side is disposed on a back side of the Bluetooth body temperature thermometer main body, and the first adhesive strap loop side is disposed on a corresponding position on the arm belt.

3. The infant body temperature and sleep monitoring system as in claim 1, wherein the second adhesive strap comprises a second adhesive strap hook side and a second adhesive strap loop side; the second adhesive strap hook side is disposed adjacent to the second adhesive strap loop side; the second adhesive strap hook side is disposed at an outer side.

4. The infant body temperature and sleep monitoring system as in claim 2, wherein the second adhesive strap comprises a second adhesive strap hook side and a second adhesive strap loop side; the second adhesive strap hook side is disposed adjacent to the second adhesive strap loop side; the second adhesive strap hook side is disposed at an outer side.

5. The infant body temperature and sleep monitoring system as in claim 1, wherein the Bluetooth body temperature thermometer main body comprises a Bluetooth module, a temperature sensor, an accelerometer, a Bluetooth antenna and a battery; the temperature sensor, the accelerometer, the Bluetooth antenna and the battery are respectively electrically connected to the Bluetooth module; the temperature sensor and the accelerometer transmit measuring data obtained to the Bluetooth module; the Bluetooth module processes the measuring data obtained and then transmits the processed measuring data to the Bluetooth receiving member via the Bluetooth antenna; the battery supplies power to the Bluetooth module.

6. The infant body temperature and sleep monitoring system as in claim 5, wherein the first side of the arm belt is disposed with a pocket for receiving the Bluetooth antenna.

7. The infant body temperature and sleep monitoring system as in claim 5, wherein the Bluetooth antenna is connected to the Bluetooth module via an RF connector.

8. The infant body temperature and sleep monitoring system as in claim 5, wherein the battery is removable battery.

9. The infant body temperature and sleep monitoring system as in claim 8, wherein the battery is button battery.

10. The infant body temperature and sleep monitoring system as in claim 5, wherein the Bluetooth body temperature thermometer main body further comprises a main body outer casing; the Bluetooth module, the accelerometer and the battery are disposed inside the main body outer casing; the temperature sensor has a probe which is disposed on an outer side of the main body outer casing; the Bluetooth antenna extends from interior of the main body outer casing to exterior of the main body outer casing.

11. The infant body temperature and sleep monitoring system as in claim 1, wherein the Bluetooth body temperature thermometer main body has a height of 5-10 mm.

12. The infant body temperature and sleep monitoring system as in claim 1, wherein the arm belt is made of highly permeable Nylex fabric for medical use.
